(19) United States
(12) Patent Application Publication
(10) Pub. No.: US 2026/0216485 A1
(43) Pub. Date: Jul. 30, 2026

(54) DEVICE FOR GUIDEWIRE INSERTION AND METHOD FOR ITS USE

(71) Applicant: South 53 LLC, San Juan Capistrano, CA (US)

(72) Inventors: Hernán Rebolledo Berrio, Punta Arenas (CL); Rodney Brenneman, San Juan Capistrano, CA (US)

(21) Appl. No.: 19/457,871

(22) Filed: Jan. 23, 2026

Related U.S. Application Data
(60) Provisional application No. 63/750,179, filed on Jan. 27, 2025.

(51) Int. Cl. A61M 25/09; A61M 25/01; A61M 25/06

(52) U.S. Cl. CPC A61M 25/09041; A61M 25/0108; A61M 25/065; A61M 25/0693; A61M 2025/09116; A61M 2205/583

(57) ABSTRACT

An insertion device includes a needle assembly having a distal end with a needle tip and a needle connector, a handle portion releasably connectable to the needle assembly, the handle portion including a handle connector portion that is configured to mate with the needle connector. A guidewire tube is connected to the handle portion, and is coiled to store a guidewire. The guidewire tube has an uncoiled length that is longer than its coiled circumference. The coil of the guidewire tube and the handle portion generally extend along a common plane. An axis through a center of the coil of the guidewire tube is substantially perpendicular to the common plane.

### CROSS-REFERENCE TO RELATED APPLICATION(S)

This patent application claims the benefit of priority from U.S. Provisional Patent Application Ser. No. 63/750,179, filed Jan. 27, 2025, titled “DEVICE FOR GUIDEWIRE INSERTION AND METHOD FOR ITS USE,” which is incorporated herein by reference in its entirety.

### FIELD

This application relates generally to devices and methods for providing access to interior spaces of a patient and more particularly to such devices and methods that operate by introducing a guidewire into a target region of the body that can be used to facilitate introduction of a lumen such as a catheter.

### BACKGROUND

Patient care may require vascular access to facilitate various procedures including, for example, administration of drugs, fluids, or parenteral nutrition, blood sampling, blood pressure monitoring including central venous pressure or interlocking pressure in the pulmonary artery, PICC lines, or ports. Such access may be, for example, to a jugular or peripheral vein, to include those in the neck, chest, arm or groin, and may be peripheral or central, or central lines placed from a peripheral vein. Insertion of such lines is often performed by use of variations of the Seldinger technique, in which a hollow needle is used to introduce a guidewire into the vein. A physician may use the presence of fluids flowing from the patient through the hollow needle, to confirm that the correct body part has been accessed by the needle. A syringe attached to the needle may be used to provide suction to promote flow of fluid from low pressure systems such as veins. Following removal of the syringe from the hollow needle, a guidewire is threaded through the needle and left in place while the needle is removed. using a needle through which the guidewire is threaded.

At this point, a catheter to be inserted is passed over the guidewire to properly position it in the target vessel. After insertion of the catheter, the guidewire may be removed, leaving the catheter in place.

In placing the needle, radiography or ultrasound guidance may be used to ensure that the needle is in the target vein and has not penetrated too little or too far. In this regard, the needle may include coatings, materials, and/or structures that provide improved imaging contrast.

Typical devices and methods for the Seldinger technique require two handed operation, during disconnect of the syringe and threading of the guidewire, which can raise issues with maintaining quality imaging of the needle in the patient. If the user is using one hand to operate the imaging device and the other to ensure proper placement and orientation of the needle, coupling or uncoupling portions of the insertion device becomes difficult. Typically, such an uncoupling step would be performed, for example, between the step of placing the needle in the correct location and passing the guidewire though the needle. For example, where a syringe is used to provide suction, the syringe may be removed and replaced with a guidewire once the location of the needle end has been established as correct.

The following disclosure relates generally to a device and method for the insertion of a guidewire into a target body space. While veins are the primary application, there may be other applications such as insertion into a body cavity, plural space, organ, artery or vein to which it may be equally applicable. In general, the devices and techniques and their various modifications are applicable to both human and veterinary patients.

Thus, a need exists for improved systems and methods for insertion of catheters.

### SUMMARY

In an embodiment, an insertion device includes a needle assembly having a distal end with a needle tip and a needle connector, a handle portion releasably connectable to the needle assembly, the handle portion comprising a handle connector portion that is configured to mate with the needle connector, and a guidewire tube connected to the handle portion, the guidewire tube coiled to store a guidewire, the guidewire tube having an uncoiled length that is longer than its coiled circumference. The coil of the guidewire tube and the handle portion generally extend along a common plane. An axis through a center of the coil of the guidewire tube is substantially perpendicular to the common plane.

In an embodiment, an insertion device includes a needle assembly having a distal end with a needle tip and a needle connector, a handle portion releasably connectable to the needle assembly, the handle portion comprising a handle connector portion that is configured to mate with the needle connector. At least one of the needle connector and the handle connector is sufficiently transparent such that a fluid present therein is visible. The insertion device includes a guidewire tube connected to the handle portion, the guidewire tube coiled to store a guidewire.

In an embodiment, an insertion device includes a needle assembly having a distal end with a needle tip and a needle connector, a handle portion releasably connectable to the needle assembly, the handle portion comprising a handle connector portion that is configured to mate with the needle connector, and a guidewire tube connected to the handle portion, the guidewire tube coiled to store a guidewire, a diameter of coils of the coiled guidewire tube is in the range between 2 inches and 4 inches.

An insertion device includes a needle assembly having a distal end with a needle tip and a needle connector, a handle portion releasably connectable to the needle assembly, the handle portion comprising a handle connector portion that is configured to mate with the needle connector, and at least one of the needle connector and the handle connector having a vented chamber formed therein to maintain ambient pressure in the vented chamber, and a guidewire tube connected to the handle portion. The insertion device comprises a transparent or translucent region to provide a visual indication of blood entering such region.

In an embodiment, a method of inserting a catheter into a target body cavity includes using an insertion device as described herein.

In an embodiment, an insertion device comprises a needle assembly having a distal end with a needle tip and a needle connector, a frame portion configured to be manually engaged by a user and releasably connectable to the needle assembly, the frame portion comprising a frame connector portion that is configured to mate with the needle connector, a guidewire tube connected to the frame portion, the guidewire tube coiled to store a guidewire, and an echogenic structure disposed on the needle assembly at a predetermined distance from the needle tip. The echogenic structure comprises a bulb having an asymmetric shape along a longitudinal axis of the needle assembly, The bulb having a proximal end and a distal end, the distal end being closer to the needle tip than the proximal end. The widest diameter portion of the bulb is closer to the proximal end than the distal end.

In an embodiment, an insertion device comprises a needle assembly having a distal end with a needle tip and a needle connector; a frame portion configured to be manually engaged by a user and releasably connectable to the needle assembly, the frame portion comprising a frame connector portion; a guidewire tube connected to the frame portion, the guidewire tube coiled to store a guidewire; and a locking collar that is configured to releasably connect the needle connector and the frame connector portion. The locking collar is configured to constrain relative rotation between the frame connector portion and the needle connector so as to maintain the needle assembly in a predetermined rotational orientation relative to the frame during use.

In an embodiment, an insertion device comprises a needle assembly having a distal end with a needle tip and a needle connector; a frame portion configured to be manually engaged by a user and releasably connectable to the needle assembly, the frame portion comprising a frame connector portion configured to mate with the needle connector; and a guidewire tube connected to the frame portion, the guidewire tube coiled to store a guidewire, a diameter of coils of the coiled guidewire tube is between 2 inches and 4 inches. The frame has a maximum thickness portion along a plane perpendicular to guidewire. The guidewire tube lies within an imaginary thick plane defined by the maximum thickness portion.

In an embodiment, an insertion device comprises a needle assembly having a distal end with a needle tip and a needle connector; a frame portion configured to be manually engaged by a user and releasably connectable to the needle assembly, the frame portion comprising a frame connector portion configured to mate with the needle connector; and a guidewire tube connected to the frame portion, the guidewire tube coiled to store a guidewire. The frame portion includes an open portion between the frame connector portion and the proximal end of the frame portion. The open region is configured to allow for a user to pinch the guidewire between a user's thumb and a user's finger for controlled advancement or retraction of the guidewire.

### BRIEF DESCRIPTION OF THE DRAWINGS

The patent application may be better understood with reference to the drawings:

FIG. 1 is a partially exploded left side elevation view of an insertion device for advancing a guidewire in accordance with an embodiment.

FIG. 2 is a front elevation view of a portion of the device of FIG. 1 in accordance with an embodiment.

FIG. 2A is a detail view of a portion 2A of FIG. 2 in a partial sectional view.

FIG. 2B is a front elevation view of a portion of the device of FIG. 1 in accordance with another embodiment.

FIG. 3 is top plan view of the embodiment of FIG. 1.

FIG. 3A is partial sectional front elevation view along the line 3A-3A as shown in FIG. 3.

FIG. 3B is a detail view of a portion of FIG. 3A.

FIG. 4 shows an open region, between a frame connector portion and a proximal end of a frame portion, the open region is configured to allow for a user to pinch the guidewire between a user's thumb and a user's finger for controlled advancement or retraction of the guidewire.

FIG. 5 shows two exemplary needle assemblies, the left needle assembly having a symmetrical echogenic structure thereon and the right needle assembly having an asymmetrical echogenic structure thereon.

FIG. 6 shows two exemplary needle assemblies, each needle assembly has an asymmetrical echogenic structure thereon.

FIG. 7 shows needle assembly, FIG. 7(a) and 7(b) show the needle assembly with and without a protective sheath thereon, and FIG. 7(c) shows an exploded view of the asymmetrical echogenic structure on the needle assembly.

FIG. 8 shows a graphical representation of the penetration of a silicone sheet skin simulation using the needle assembly with the asymmetrical echogenic structure, FIG. 8 shows the changes in force as the needle tip punctures and the asymmetrical echogenic structure interacts with and passes through the skin or vessel wall.

FIG. 9 shows a perspective view of an insertion device for advancing a guidewire in accordance with an embodiment.

FIG. 10 is partial sectional view along the line 10-10 as shown in FIG. 9.

FIGS. 11A-11D show variations on needles with one or more bulbs to enhance (increase) echogenicity and/or haptics.

### DETAILED DESCRIPTION

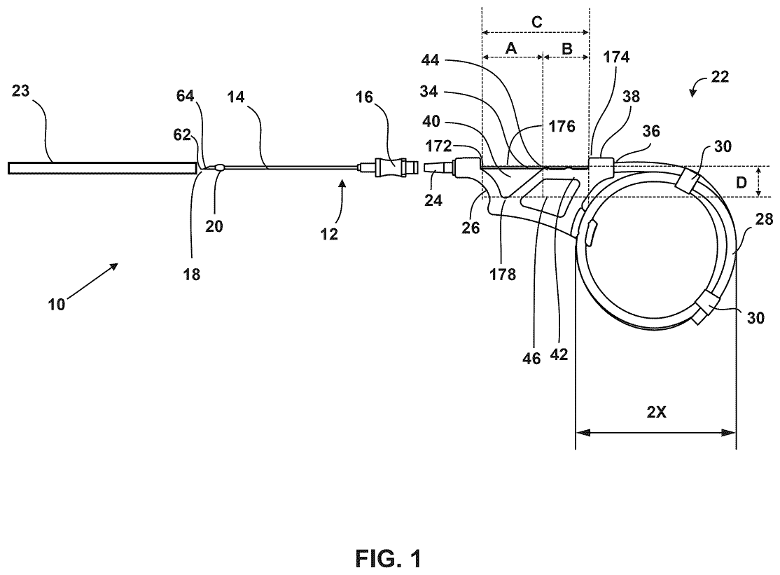

FIG. 1 is a partially exploded front elevation view of an insertion device 10 that is configured to be used to insert a guidewire into a target region in a patient's body. The target region may be, for example, a body cavity or an interior space in a hollow organ, pleural space, blood vessel (artery or vein), bladder, or intestine. For the purposes of this disclosure the discussion will focus on the application involving a patient's vein, but the scope of the disclosure should not be considered to be so limited.

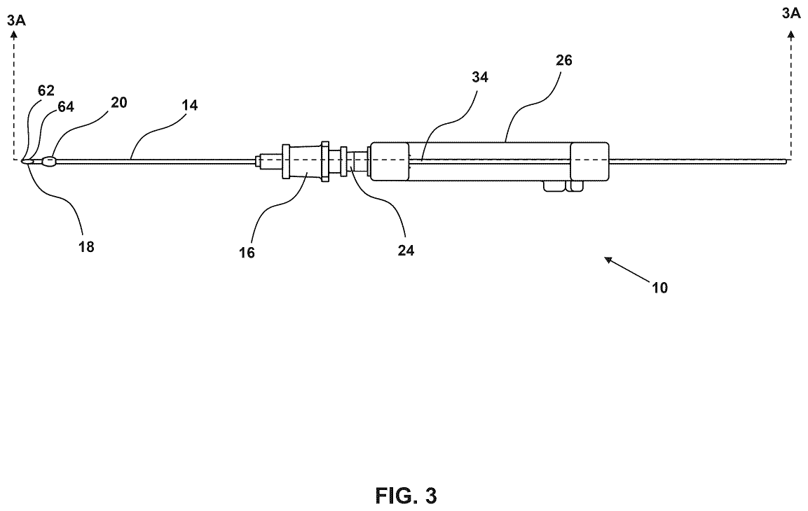

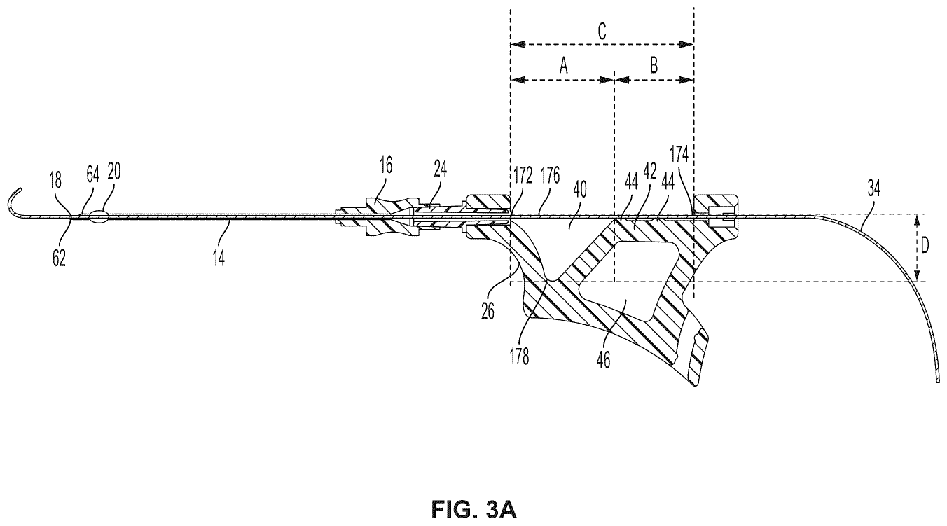

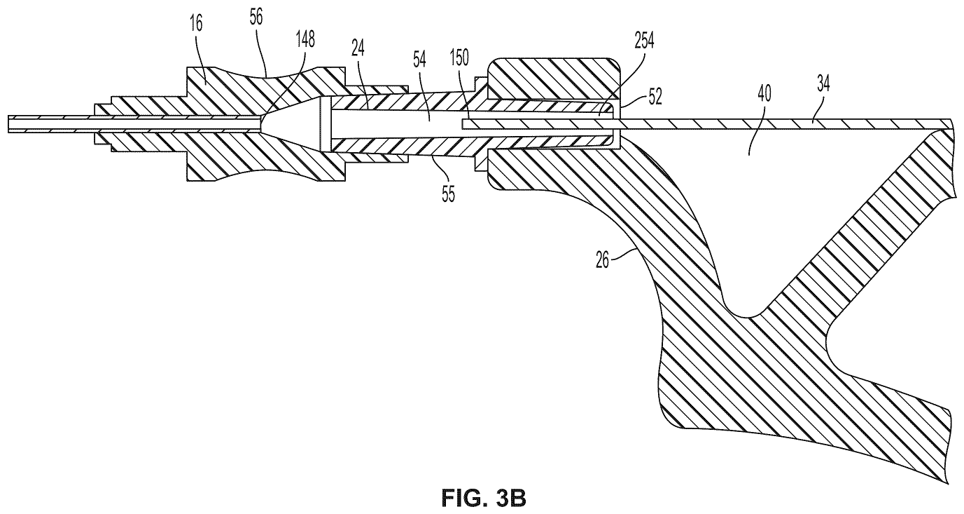

The insertion device 10 includes a needle assembly 12 that includes a needle 14. The needle 14 includes a first end 148 and a second, sharp end 18 as shown in FIGS. 3A and 3B. In one embodiment, the first end of the needle 14 may interchangeably referred to as a proximal end of the needle 14, and the second end of the needle 14 may interchangeably referred to as a distal end of the needle 14. In an embodiment, the needle assembly 12 has a distal end with the needle tip 18 and a needle connector 16. In an embodiment, the needle assembly 12 has a proximal end with the needle connector 16. In an embodiment, when the needle assembly 12 is connected to a frame/handle portion 22, the distal end of the needle assembly 12 is facing away from the frame portion 22, and the proximal end of the needle assembly 12 is facing towards from the frame portion 22. In an embodiment, when the needle assembly 12 is connected to the frame portion 22, the proximal end of the needle assembly 12 is facing towards a distal end of the frame portion 22.

As will be discussed in greater detail below, proximate the sharp end 18, the needle 14 may include an echogenic structure 20 that is configured to improve an imaging contrast of the needle during an imaging process.

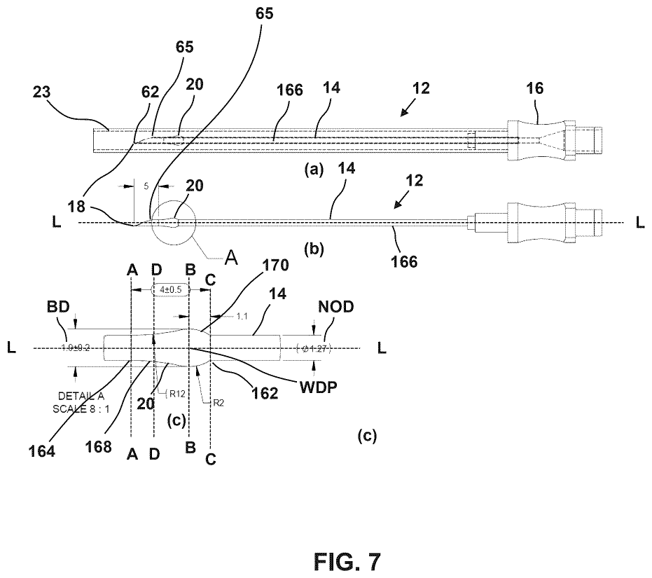

The details of the echogenic structure 20 will be described with respect to FIG. 7. In an embodiment, as shown in FIG. 7, the insertion device 10 comprises the echogenic structure 20 disposed on the needle assembly 12 at a predetermined distance from the needle tip 62. As shown in FIG. 7(b), at least a portion (e.g., a distal end 164) of the bulb 20 is disposed at a predetermined distance of 5 mm from the needle tip 62. In an embodiment, the predetermined distance between the distal end 164 of the bulb 20 and the needle tip 62 is between 1 mm and 5 mm. In an embodiment, the predetermined distance between the distal end 164 of the bulb 20 and the needle tip 62 may be +/−20%, +/−10%, or +/−5% of the values/ranges described herein. FIG. 7(a)-7(c) show the needle 14 with bulb 20 in accordance with one embodiment (e.g., which can be used for central venous access). In one embodiment, the needle tip is angled/beveled, so the needle 14 may have proximal and distal ends 65 of the tip.

In one embodiment, the echogenic structure 20 comprises an asymmetric shape along a longitudinal axis of the needle assembly 12. In an embodiment, the echogenic structure 20 is a tapered echogenic structure 20 having a tapered configuration. Specifically, the bulb 20 has a proximal end 162 and the distal end 164. The distal end 164 is closer to the needle tip 62 than the proximal end 162. The widest diameter portion WDP of the bulb 20 is closer to the proximal end than the distal end 164. The diameter of the bulb 20 used to measure the widest diameter portion WDP is taken at cross section across a plane perpendicular to the longitudinal axis of the needle shaft 166.

In one embodiment, the bulb 20 has a diameter that is between 1.7 and 2.1 mm at the widest diameter portion WDP perpendicular to the longitudinal axis L-L of the needle shaft/assembly. In one embodiment, the bulb 20 has a maximum diameter that is approximately 1.9 mm at the widest diameter portion WDP perpendicular to the longitudinal axis L-L of the needle shaft/assembly. In an embodiment, the maximum diameter of the bulb may be +/−20%, +/−10%, or +/−5% of the ranges described here.

In various embodiments, the maximum diameter of the bulb 20 is disposed at a distance in the range between 50% and 85% of the total bulb length, from the proximal end 162 of the bulb 20. In one embodiment, the maximum diameter of the bulb 20 is disposed at a distance of 1.1 mm from the proximal end 162 of the bulb 20. In an embodiment, the distance may be +/−20%, +/−10%, or +/−5% of the ranges described here. In one embodiment, the widest diameter portion WDP of the bulb 20 is shaped and dimensioned to engage with tissue portions around a target body region such that the force to retract the needle shaft 166 is greater than the force to advance it distally toward the target body region. The target body region may be a low-pressure region, such as a vein.

In an embodiment, the diameter of the distal end 164 of the bulb is slightly greater than (increasing by a fraction of a millimeter of the bulb material) the diameter of the needle shaft 166 around which the distal end 164 of the bulb 20 is formed. In an embodiment the diameter of the proximal end 162 of the bulb 20 is slightly greater than (increasing by a fraction of a millimeter of the bulb material) the diameter of the needle shaft 166 around which the proximal end 162 of the bulb 20 is formed. In one embodiment, the bulb may be about 2.8 mm to about 3.2 mm long, for example, about 3 mm long. At the proximal end of the bulb 20 (nearest to the operator's hand), the transition to the needle shaft is relatively steep, so the maximal bulb diameter (e.g., in one embodiment this is between about 1.6 mm to 2.0 mm, for example about 1.8 mm) is between 0.5 mm and 1.0 mm from the proximal end of the bulb 20. By contrast, the distal end of the bulb 20 (nearest the needle tip), may be greater than 1.0 mm from the maximum bulb diameter and may be in the range between 2.0 mm and 2.5 mm. In one embodiment, the bulb may have a diameter of between 1.5 and 2.0 times the needle diameter. In one embodiment, the bulb for an 18 GA 1.25 mm diameter needle would therefore be in the range between 1.8 mm to 2.5 mm diameter. In one embodiment, the bulb length may be in the range between 2.0 and 3.0 times the needle diameter. In one embodiment, the bulb length may be 2.5 times the needle diameter. In one embodiment, a symmetric bulb 20 may have the maximal bulb diameter midway (50%) along its length, whereas an asymmetric tapered bulb may have the maximal diameter shifted proximally to between 20% and 40% of the bulb length.

In an embodiment, the bulb 20 has an overall length between 3 mm and 5 mm measured along the longitudinal axis of the needle assembly. In an embodiment, the bulb 20 has an overall length between 3.5 mm and 4.5 mm measured along the longitudinal axis of the needle assembly. In an embodiment, the bulb has an overall length of 4 mm measured along the longitudinal axis of the needle assembly. In an embodiment, the overall length of the bulb may be +/−20%, +/−10%, or +/−5% of the ranges described here.

In an embodiment, a ratio of the overall length of the bulb 20 to the maximum diameter of the bulb 20 is in the range between 1.25 and 2.00. In one embodiment, the ratio of bulb length to diameter may be in the range between 1.25 and 1.80. In one embodiment, the ratio of bulb length to diameter may be between 1.65 and 1.75. In an embodiment, the ratio may be +/−20%, +/−10%, or +/−5% of the ranges described here.

The bulb has a ratio of length greater than diameter describing an ovalized profile. When the location of the largest diameter is shifted towards one end, an asymmetric ovalized shape is formed. This patent application includes symmetric shaped bulb and/or asymmetric shaped bulbs, across a range of possible needle sizes and corresponding (scaled) bulb sizes.

Referring to FIG. 7, in one embodiment, the length of the bulb 20 is measured between a first end 164 of the bulb at a line A-A and a second end 162 of the bulb at a line C-C. That is, the first end 164 of the bulb 20 is at the line A-A and the second end 162 of the bulb 20 is at the line C-C. In one embodiment, the length of the bulb 20 is measured along the longitudinal axis L-L of the needle assembly 12. In one embodiment, the length of the bulb 20 may be 3.5 mm. In one embodiment, the length of the bulb 20 may be between 3.5 mm and 4 mm. In one embodiment, the length of the bulb 20 may be 4 mm. In one embodiment, the length of the bulb 20 may have tolerance of +/−0.5 mm.

In one embodiment, the diameter of the bulb 20 at the line A-A may be different from the diameter of the bulb 20 at the line C-C. In one embodiment, the diameter of the bulb 20 at the line A-A is same as the diameter of the bulb 20 at the line C-C. As shown in FIG. 7, the bulb 20 may have a radius of R2 at the line B-B and a radius of R12 at line D-D. In one embodiment, the radius R2 of the bulb is larger than the radius R12 of the bulb.

In one embodiment, the diameter of the needle 14 is in the range between 1 mm and 1.5 mm. In one embodiment, the diameter of the needle 14 is in the range between 1.1 mm and 1.4 mm. In one embodiment, the diameter of the needle 14 is in the range between 1.2 mm and 1.3 mm. In one embodiment, the diameter of the needle 14 is 1.27 mm. In an embodiment, the diameter of the needle 14 may be +/−20%, +/−10%, or +/−5% of the values/ranges described here.

In one embodiment, the distance between the line B-B (at which the bulb 20 has the radius R2) and the line C-C (at the second end 162 of the bulb 20) is in the range of 0.85 mm and 1.35 mm. In one embodiment, the distance between the line B-B (at which the bulb 20 has the radius R2) and the line C-C is 1.1 mm. The maximum diameter of the bulb 20 is disposed at 1.1 mm from the line C-C or the second end 162 of the bulb 20.

In one embodiment, the diameter of the bulb 20 at the line B-B is the maximum diameter of the bulb 20. In one embodiment, the diameter of the bulb 20 at the line B-B is in the range between 1.6 mm and 2.2 mm. In one embodiment, the diameter of the bulb 20 at the line B-B is in the range between 1.8 mm and 2.0 mm. In one embodiment, the diameter of the bulb 20 at the line B-B is 1.9 mm. In one embodiment, the diameter of the bulb at the line B-B has a tolerance of +/−0.2 mm.

In an embodiment, the echogenic structure 20 is made of a material having an echogenicity different from an echogenicity of a material of the needle shaft 166. In an embodiment, the bulb 20 is formed from a UV-cured polymeric material containing echogenic particles selected from the group consisting of minerals including diamond, titanium dioxide or precious stones like ruby or garnet, or metals including tungsten, stainless steel, and cobalt chrome.

In an embodiment, the diameter of the bulb 20 slowly/progressively increases from the distal end 164 to the proximal end 162. In an embodiment, as shown in FIG. 7, the bulb 20 includes a straight taper or a concave shaped portion 168 and a convex shaped portion 170. In an embodiment, as shown in FIG. 7, the radius R12 of the bulb defines the concave shaped portion 168 and the radius R2 of the bulb defines convex shaped portion 170. In an embodiment, the bulb 20 transitions from the concave shaped portion 168 (e.g., having the radius R12) to the convex shaped portion 170 (e.g., having the radius R2) at the maximum diameter of the bulb 20. In one embodiment, the convexed radius R2 may be in the range between 0.2 mm and 0.5 mm. In one embodiment, the concaved radius R12 may be in the range between 0.8 mm and infinity (i.e., a straight line).

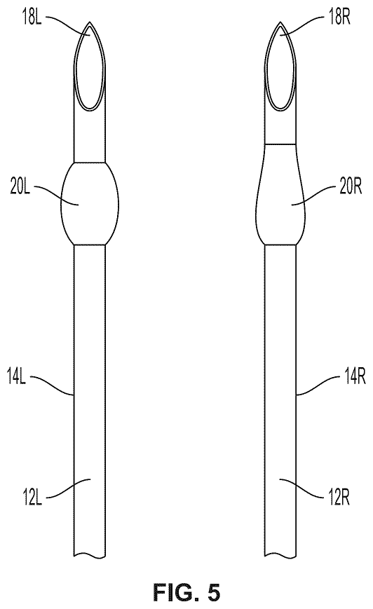

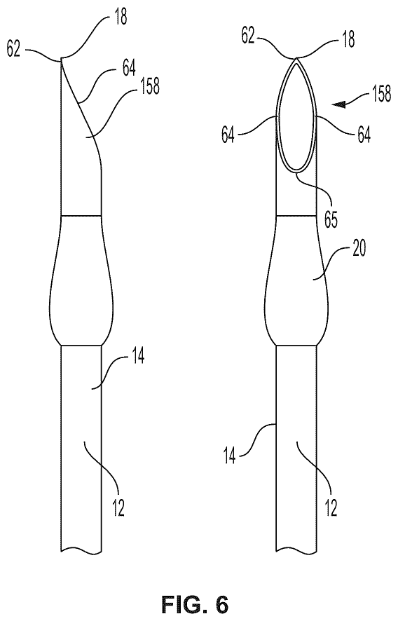

FIG. 5 shows two exemplary needle assemblies 12 and 12, the left needle assembly 12L having a symmetrical echogenic structure 20L thereon and the needle tip 18L and the right needle assembly 12R having an asymmetrical echogenic structure 20R thereon and the needle tip 18R. FIG. 5 shows the ‘asymmetric’ or tapered echogenic bulb and comparing it to the symmetric bulb described in other embodiments of this patent application. FIG. 6 shows two exemplary needle assemblies 12, each needle assembly 12 has an asymmetrical echogenic structure 20 thereon.

The needle 14 may be, for example, a stainless steel surgical needle including an interior lumen or channel extending therethrough. In an embodiment, the needle may be an 18 gauge needle, but as will be appreciated other sizes may be selected. The needle connector 16 may be a plastic connector, including, but not limited to, a Luer lock or Luer slip style connector that may be releasably connected to a handle portion 22 of the insertion device 10. In an embodiment, a portion of or all of the needle connector 16 may be made from a material that is optically transparent or translucent, such that the user can visualize any fluid flow through the needle. In the case of the target being a blood vessel, blood flow visible through the connector can be an indicator that the placement of the needle tip within a target vessel is correct. A needle tip portion 158 may include a pointed end 62 and two beveled sides 64 as shown in FIGS. 1, 3, 3A, and 6. In one embodiment, the needle tip 18 may include the pointed end 62. In another embodiment, the needle tip 18 may include the pointed end 62 and the two beveled sides 64. In such an embodiment, the needle tip 18 may be interchangeably referred to as the needle tip portion 158.

The needle assembly 12 includes a needle shaft 166 (as shown in FIG. 7) extending between the needle tip 18 and the needle connector 16. The needle shaft 166 is configured to receive the guidewire 34 therethrough. The bulb 20 is disposed on the needle shaft 166 between the needle tip 18 and the needle connector 16.

In an embodiment, the handle portion 22 is configured to be manually engaged by a user and releasably connectable to the needle assembly 12. In an embodiment, the handle portion 22 is releasably connectable to the needle connector 16 at the proximal end of the needle assembly 12.

The handle portion 22 includes a handle connector 24 (e.g., at a distal end of the handle portion 22) that is configured to mate with the needle connector 16. As will be appreciated, the mating between the connectors may be achieved through a variety of available approaches, including for example a friction or friction interference fit, or a threaded connection. In an embodiment, the connectors may be configured as a Luer lock or a Luer slip type.

In one embodiment, the handle portion 22 and the frame portion 22 may be interchangeably used. The frame portion 22 may be configured to be manually engaged by a user. The tube may be part of the handle or frame portion 22. Either the handle portion 22 or the frame portion 22 works and may be used in the detailed description below, but that the “handle” should be broadly construed to be either the full structure or part of the structure that the user will engage manually for use. In one embodiment, the handle connector 24, the handle connector portion 24, the frame connector 24 and the frame connector portion 24 may be interchangeably used.

In an embodiment, when the frame portion 22 is connected to the needle assembly 12, a distal end of the frame portion 22 is facing towards the needle assembly 12, and the proximal end of the frame portion 22 is facing away from the needle assembly 12. In an embodiment, when the frame portion 22 is connected to the needle assembly 12, the distal end of the frame portion 22 is facing towards the proximal end of the needle assembly 12.

When not in use, a protective sheath 23 may be placed over the needle 14 such that the sharp end 18 is not exposed.

The handle portion 22 is made up of two main components, an advancer frame 26 and a guidewire tube 28. In an embodiment, the guidewire tube 28 is connected to the frame portion 22. In an embodiment, the guidewire tube 28 is connected to a proximal end of the frame portion 22. In an embodiment, when the guidewire tube 28 is connected to the frame portion 22, a distal end of the guidewire tube 28 is facing towards the frame portion 22. In an embodiment, when the guidewire tube 28 is connected to the frame portion 22, the distal end of the guidewire tube 28 is facing towards the proximal end of the frame portion 22. In an embodiment, the guidewire tube coiled 28 is configured to store a guidewire 34.

The advancer frame 26 may be made from a plastic material such as polyester or HDPE and may in principle be manufactured by additive manufacturing, injection molding, hybrid, or other plastic manufacturing techniques.

The guidewire tube 28 may be an HDPE tubing material and may be clear, opaque, or translucent. In an embodiment, the guidewire tube 28 is heat formed into the coil shape that is shown in the FIGS. Additionally, guidewire bands 30 may be included to hold the coiled shape of the guidewire tube 28. The guidewire bands 30 may be plastic or non-woven tape material, or may be in the form of a plastic or metal clip, for example. Additionally, the advancer handle frame 26 may include a notch or clip 32 that attaches the guidewire tube 28 to the advancer handle frame 26.

The guidewire 34 is partially contained within the guidewire tube 28 and extends through an end 36 of the guidewire tube 28 into a mating portion 38 of the advancer frame 26. Passing through this mating portion 38, the guidewire 34 then crosses an open region 40 of the advancer frame 26, into and though the handle connector 24. From there, it passes though the needle connector 16 and into the channel of the needle 14.

In an embodiment, at least a portion of the open region 40 of the advancer handle frame 26 includes a platform portion 42. In an embodiment, the insertion device 10 comprises the platform portion 42 against which a user can push the guidewire 34 to advance or retract the guidewire 34. The platform portion 42 is positioned such that the user's thumb is able to engage the guidewire 34 from above and apply pressure against the platform portion 42 to advance or retract the guidewire 34.

This platform portion 42 may include one or more protrusions 44 that slidably support at least a portion of the guidewire 34 as it rests in the open region 40. In an embodiment, the one or more protrusions 44 extend in a direction perpendicular to a surface of the platform portion 42 and perpendicular to an axis of the guidewire 34. In an embodiment, the one or more protrusions 44 configured to facilitate advancement and retraction of the guidewire 34. The one or more protrusions 40 are configured to eliminate frictional contact between the guidewire 34 and the platform portion 42 by limiting an area of surface contact therebetween. The open portion 40 is configured to permit both thumb-slide actuation and pinch actuation of the guidewire 34. When not in use, a distal end of the guidewire 34 is contained within the channel of the needle 14 and does not extend past its end 18. In one embodiment, the height of the protrusions 40 may be in the range between 0.5 mm and 2.0 mm. In one embodiment, the length of the protrusions 40 may be in the range between 0.5 mm and 2.0 mm. In one embodiment, the protrusions 40 may be spaced equidistance from the ends of the platform portion 42.

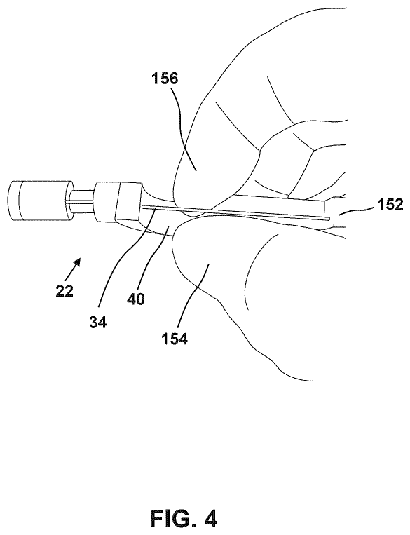

As shown in FIG. 4, the frame portion 22 includes the open portion 40 between the frame connector portion and the proximal end 152 of the frame portion 22. The open region 40 is configured to allow for a user to pinch the guidewire 34 between a user's thumb 154 and a user's finger 156 for controlled advancement or retraction of the guidewire 34. In an embodiment, the user's finger 156 is a middle finger. In an embodiment, the user's finger 156 is an index finger. In an embodiment, the guidewire 34 extends across the open portion 40 from a proximal end 152 of the frame portion 22 to the frame connector portion 24 and at least partly into the needle assembly 12.

Referring to FIGS. 1 and 3A, in an embodiment, the length A of the open region 40 is in the range of 10 mm and 30 mm. In an embodiment, the length A of the open region 40 is 20 mm. In an embodiment, the length B of the platform portion 42 is in the range of 10 mm and 30 mm. In an embodiment, the length B of the platform portion 42 is 20 mm.

In an embodiment, the length C from a distal end 172 of the open region 40 to a proximal end 174 of the platform portion 42 is in the range of 20 mm and 60 mm. In an embodiment, the length C from the distal end 172 of the open region 40 to the proximal end 174 of the platform portion 42 is 40 mm. In an embodiment, the height D from a top 176 of the open region 40 to a bottom 178 of the open region 40 is in the range of 10 mm and 30 mm. In an embodiment, the height D from the top 176 of the open region 40 to the bottom 178 of the open region 40 is 20 mm.

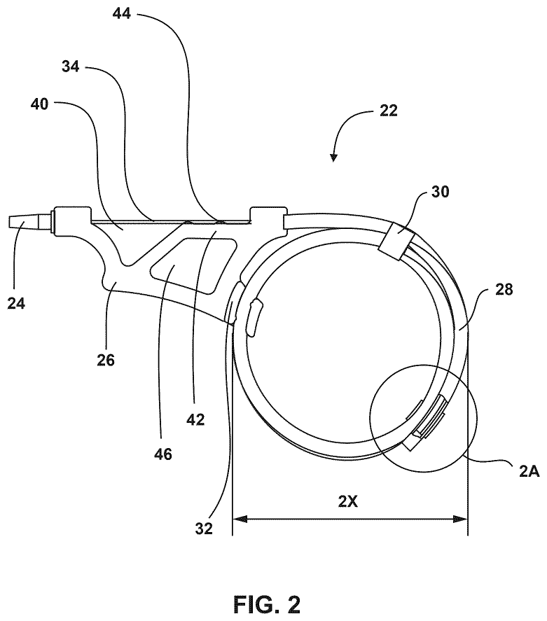

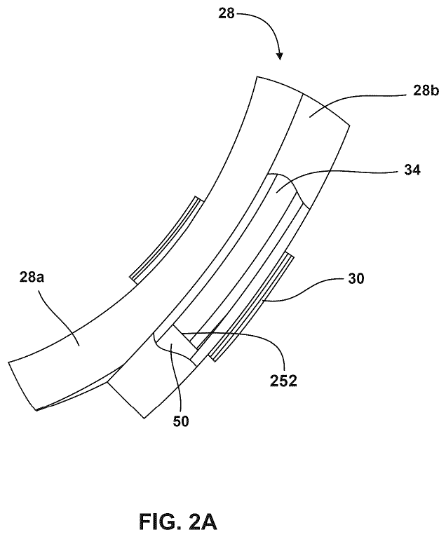

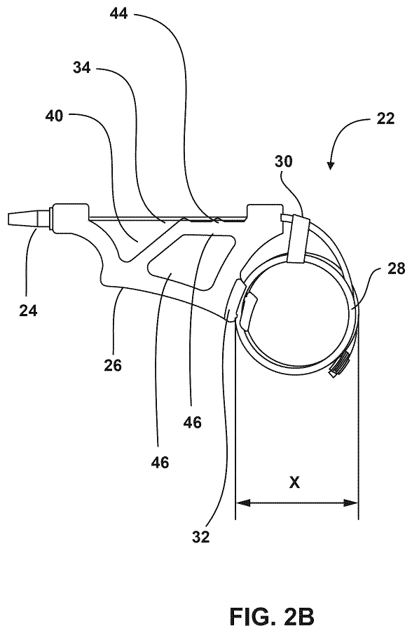

FIGS. 2, 2A, 3, 3A, and 3B generally show the same features of the insertion device 10, with different degrees of detail.

Turning now to FIG. 2, the handle portion 22 is shown in greater detail, and the reference numerals correspond substantially to the reference numerals of FIG. 1. More visible in FIG. 2 is a finger opening 46. When the user is holding the insertion device 10 in a hand, the forefinger or middle finger can be placed through the finger opening 46 to help steady the insertion device 10. While the finger opening 46 is shown in FIGS. 1 and 2 as being a polygon with rounded corners, an optional ergonomic design could include a finger opening 46 that is circular or substantially circular.

FIG. 2A, a detail of FIG. 2 illustrates a portion of the guidewire tube 28 in a partial sectional view. As shown, a first portion 28a and a second portion 28b overlap where the tube coils overlap. A guidewire band 30 is shown in partial sectional view at the overlap position. The guidewire 34 is shown within the guidewire tube 28, along with a stop 50. The stop 50 may be, for example, a piece of tubing that has a diameter sufficiently small that a distal end 252 of the guidewire 34 cannot pass through it. The stop 50 may likewise be in the form of a plug at the end of the guidewire tube 28.

In operation, the guidewire bands 30 securely hold the coils of the guidewire tube 28 together, as well as provide a secure connection to the handle portion 22. As a result, the insertion device 10 (i.e., the needle assembly 12, the handle portion 22, and the guidewire tube 28) can be held and controlled as a unit using a single hand. In this regard, the coils of the guidewire tube 28 may be joined to the handle portion 22 such that they are aligned along an axis thereof (and a common axis with the needle assembly 12) and opposite to an open bevel portion of the needle 14 (i.e., sharp end 18). Given this substantially unitary assembly with major components aligned along a common axis, may allow the operator to infer in what direction the needle bevel or sharp end 18 is pointed.

Surgical guidewires suitable for use as guidewire 34 may be solid or wrapped or braided wire. For example, steel or nitinol solid wires are sometimes used, while wires made with a solid steel or nitinol core with a smaller diameter wire wrapped around the core are also typical. The specific properties of the wire, pushability, steerability, and imaging characteristics, help to select the desired guidewire construction. For example, where imaging will be using radiographic techniques, radio-opacity or radiolucency may be a key feature, whereas in ultrasound imaging the echogenicity of the material may be. In an embodiment, the guidewire 34 is selected to have a diameter of approximately 0.035 inches (approximately 1 mm or slightly less). As will be understood, the diameter of the guidewire 34 is dependent at least in part on the selected needle gauge.

In general, such guidewires are available in standard lengths of 50 cm or 80 cm. However, as will be appreciated, the applicability of the devices and methods described herein do not depend on using these specific lengths, and more generally, the guidewire 34 may be between 50 cm and 80 cm and more particularly between 50 cm and 70 cm. These lengths tend to be suitable for a device that can be held in the palm of a user's hand. A much longer guidewire 34 can make compact size difficult to achieve, while a much shorter one can cause the guidewire to be insufficiently long to be advanced into the cavity without the distal end inadvertently exiting through the open region 40. The open portion 40 and the open region 40 are interchangeably used in this application.

As may be seen from the FIGS, the device 10 is substantially symmetrical, which means that the insertion device 10 may be used with either the right or left hand, depending on the user's preference. As may be appreciated, in general, manipulation of the needle 14 is more critical than manipulation of the imaging device, and thus the user will tend to prefer to use the dominant hand for holding and manipulating the insertion device 10 and the off hand for controlling the imaging device for visualization-aided placement of the needle sharp end 18. Similarly, the inventors have determined that the insertion device 10 may be comfortably held with either of an overhand or pen grip.

The echogenic structure 20 may be made from materials selected to enhance echogenic properties of the needle end. That is, when ultrasound imaging is used to assist in guiding the surgical procedure, the location of the echogenic structure can be seen by the surgeon more easily in the image. Appropriate materials may include, for example, selected metals, polymeric materials, ultraviolet cured materials, two-part epoxy. Additionally, secondary materials may be mixed into, or incorporated into, the echogenic structure to further improve echogenicity. These secondary materials may include enhancing materials in the form of powders, particles, beads or structures made from tungsten, stainless steel, natural or synthetic diamond, or minerals such as titanium dioxide and/or calcium. Additionally or alternately, reflective textures such as diffraction gratings may be machined or laser etched into, or placed on, the surface of the needle to improve echogenicity. Structures may also be incorporated into the bulb. For example, a reflective grid or other reflective component may be bonded to the needle shaft and subsequently encapsulated within the UV-curable epoxy or epoxy-diamond compound.

In an embodiment, a UV-cured resin such as UV-cured adhesive available from Dymax® may be applied to form the echogenic structure 20. This may be achieved by slowly turning the needle under applied UV light, while applying the adhesive to form echogenic structure 20 in the shape of a bulb. The bulb may serve a secondary purpose of acting as a stop to reduce the likelihood of over penetrating the target region. As will be appreciated, by placing the bulb at a preselected distance from the needle tip 18, the bulb will tend to resist insertion past, for example, a vein wall, to a degree greater than the preselected distance. This feature is aided where the device is applied to low pressure target regions it will pass more easily through the patient's skin than through the flexible low pressure internal structures. The preselected distance may be, for example, between 1 mm and 4 mm and more particularly between 2 mm and 3 mm. In order to facilitate this function, a bulb diameter on the order of 2 mm may be chosen, and more broadly a diameter between about 1.5 mm and 3 mm. In an embodiment, the bulb diameter ranges from 1.2 to 2 times the diameter of the needle around which it is formed. Additionally, in order to ensure that the bulb will pass well through the patient's skin, it is helpful to form it in a ramped manner such that it slopes from its maximum diameter down to a smaller diameter as shown in FIG. 3A. The overall length of the bulb may be between 1.5 and 2 times the diameter of the bulb in order to provide a bulb that is not too steeply sloped to properly enter the patient's skin. However, on the proximal side of the bulb opposite any shallow ramp, it may be advantageous to have a steeper sloping transition to the needle shaft to resist accidental retraction of the needle, thereby helping to maintain the needle tip in its desired position.

The open region 40, along with the platform portion 42 and protrusions 44 facilitate advancing and retracting the guidewire 34. In particular, one way the user can control advancing and retraction is to hold the insertion device 10 such that the user's thumb is able to engage the guidewire 34 from above, applying pressure against the platform portion 42. From this position, moving the thumb forward or backward will advance or retract the guidewire 34 respectively. The downward pressure from the thumb towards the platform portion 42 allows the user to produce enough normal force between the thumb and the guidewire 34 to produce sufficient friction between the thumb and the guidewire 34 allow the guidewire 34 to be moved. At the same time, the protrusions 44 ensure that only a portion of the guidewire 34 is in contact with the platform portion 42. This means that an overall friction force between the platform portion 42 and the guidewire 34 is reduced compared to a friction force that would be present in the absence of the protrusions 44 (i.e., if the guidewire 34 were to engage the platform portion 42 across the entire length of the platform portion 42 instead of only or primarily at the protrusions 44).

An alternate manner of advancing or retracting the guidewire 34 is to use the open region 40 to allow sufficient room for the user to pinch the guidewire 34 between a thumb and finger, typically the index or middle finger. Where the middle finger is used to hold the handle portion 22 by placing it through the finger opening 46, the index finger and thumb are well-positioned to manipulate the guidewire 34. The user then can simply advance or retract the guidewire 34 as desired. Thus, the open region 40 provides two different possibilities for control of the guidewire 34, depending on the user's preference, and/or requirements for precision.

In an embodiment, the distance between the mating portion 38 and the handle connector 24 may be in the range between 56 mm and 84 mm. In an embodiment, the distance between the mating portion 38 and the handle connector 24 may be in the range between 63 mm and 77 mm. In an embodiment, the distance between the mating portion 38 and the handle connector 24 may be in the range between 66.5 mm and 73.5 mm. In an embodiment, the distance between the mating portion 38 and the handle connector 24 may be about 70 mm.

The platform portion 42 and the open region 40 may share this dimension in any desired proportion. As a practical matter, although still workable, if either portion is below about 10 mm they will be less-well suited allowing the guidewire 34 to be advanced, because the user's fingers or thumb will have more limited traverse. In an embodiment, either portion (i.e., platform portion 42 or the open region 40) is between 10 mm and 50 mm in length. In one embodiment, the length of the open portion 40 is between 10 mm and 50 mm. In an embodiment, the length of the open portion 40 is between 20 mm and 40 mm. In an embodiment, the platform portion 42 may be omitted and the entire region may be left open. In one embodiment, from a practical perspective, the throat opening 40 for two-finger operation may be distal to the platform portion 42. The depth of the frame 22 near the open region 40 may be in the range between 5 mm and 25 mm. The depth of the frame 22 near the open region 40 may be 15 mm. In one embodiment, the depth dimension of the frame 22 near the platform region 42 and/or the finger opening 46 may be in the range between 15 mm and 25 mm. In one embodiment, the depth dimension of the frame 22 near the platform region 42 and/or the finger opening 46 may be in the range between 18 mm and 20 mm.

Where a steel-braided nitinol guidewire material is used for the guidewire 34, a j-shaped end portion (j-tip) may be included (not shown). As the j-shaped tip is advanced out of the needle, it will regain its shape, while as it is retracted, it will straighten as it enters the end of the needle.

In one embodiment, the coil of the guidewire tube 28 and the frame/handle portion 22 generally extend along a common plane. In an embodiment, the common plane may include a vertical plane that is extending along the longitudinal axis of the insertion device 10. In an embodiment, the common plane may include a vertical plane that is extending along the longitudinal axis of the needle assembly 12 and the frame portion 22 when the needle assembly 12 and the frame portion 22 are connected to each other. An axis through a center of the coil of the guidewire tube 28 is substantially perpendicular to the common plane.

In an embodiment, the guidewire tube 28 is coiled into a diameter selected to allow the device to be comfortably held in the palm of an operator's hand. In this regard, the inventors have determined that an outer diameter of about 70 mm is suitable. In an embodiment, the outer diameter may be between about 60 mm and about 100 mm.

In one embodiment, the diameter of the coiled guidewire tube is selected to fit in the palm of a user's hand. In one embodiment, the diameter of the coiled guidewire tube is in the range between 2 inches (as shown in FIGS. 2B) and 4 inches (as shown in FIG. 2). The diameter of the coiled guidewire tube 28 in FIG. 2B is around X inches, while the diameter of the coiled guidewire tube 28 in FIGS. 1 and 2 is around 2× inches. In one embodiment, the diameter of the coiled guidewire tube is in the range between 2 inches and 3 inches. In one embodiment, the diameter of the coiled guidewire tube is in the range between 2.5 inches and 3 inches. In one embodiment, the diameter of the coiled guidewire tube is under 4 inches. In one embodiment, the diameter of the coiled guidewire tube is 2 inches. In one embodiment, the diameter of the coiled guidewire tube is 2.5 inches. In one embodiment, the diameter of the coiled guidewire tube is 3 inches. In one embodiment, the diameter of the coiled guidewire tube is 3.5 inches.

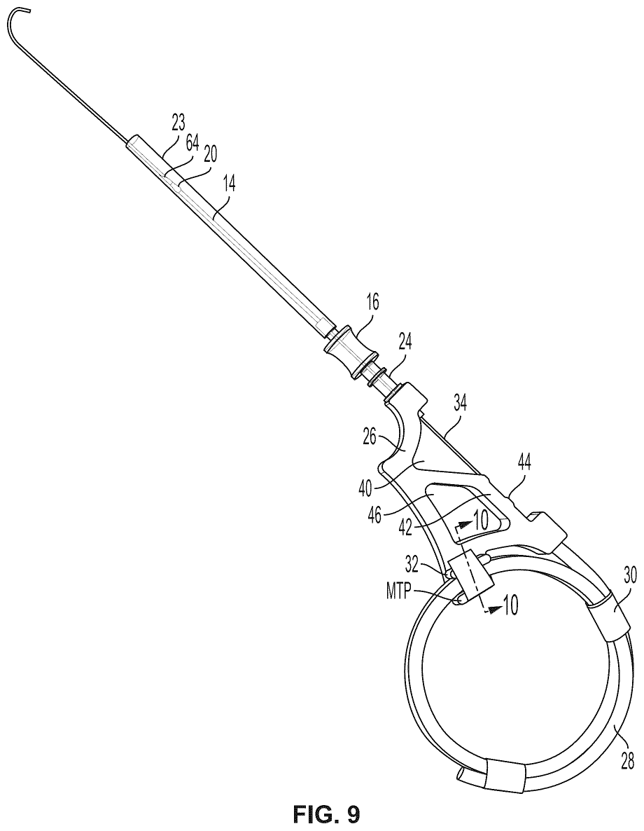

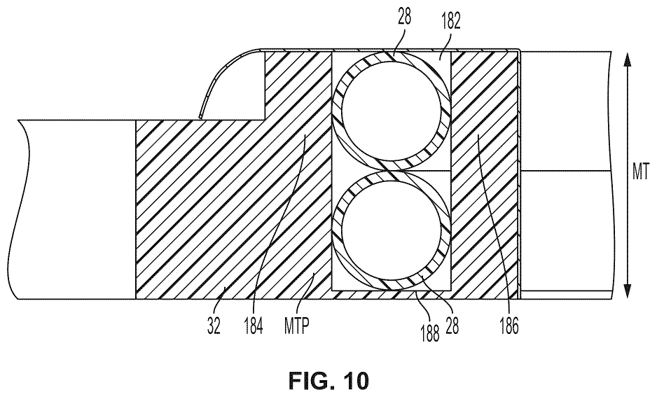

In an embodiment, the frame 22 has a maximum thickness portion MTP along a plane perpendicular to the guidewire 34. The guidewire tube 28 lies within an imaginary thick plane defined by the maximum thickness portion MTP. For example, as shown in in FIG. 10, the maximum thickness portion MTP of the frame 22 has a maximum thickness MT (e.g., extending into the paper in FIG. 9). In one embodiment, the maximum thickness portion MTP of the frame 22 includes an opening or a channel 182 that is formed between wall portions 184 and 186 and bottom/end portion 188. The wall portions 184 and the bottom/end portion 188 may be integrally formed with the frame 22. In one embodiment, the maximum thickness MT is configured to accommodate and receive at least two guidewire tubed 28 stacked (e.g., horizontally or vertically) next to each other or in side-by-side relation, optionally in direct contact with one another, as shown in FIG. 10. In one embodiment, the maximum thickness MT of the frame is in the range of 5 mm and 15 mm. In one embodiment, the dimension MT is slightly larger than the combined outer diameters of the two tubes 28 in FIG. 10. In one embodiment, MT is less than 1.3 times the combined diameters of two tubes 28.

Additionally, the overall length of the guidewire tube 28 can be selected to be shorter than a total length of the guidewire 34, and a plug or other impediment of smaller diameter than the internal diameter of the tube inserted to act as a stop to the distal end of the guidewire such that the guidewire 34 cannot be retracted beyond a selected point. The coiled plastic dispensing tube that stores the guidewire may have a plug 50 at the end farthest from the advancer handle 22, which prevents the guidewire from being over-retracted. This plug may be a small piece of silicone tubing. A tubular plug may be used because it allows ethylene oxide gas to enter the coiled dispensing tube during sterilization. The plug 50 sits just inside the end of the guidewire storage tube in FIG. 2A.

In an embodiment, the length of the guidewire tube 28 plus the length of the advancer handle frame 26 along an axis of the guidewire 34 can be selected to be at least slightly shorter than the length of the guidewire 34 such that the guidewire 34 is not accidentally retracted past the needle connector 16 and handle connector 24. For example, the length of the advancer handle frame 26 as measured between the mating portion 38 and handle connector 24 plus the length of the guidewire tube 28 is less than a length of the guidewire 34. This will assist the user in preventing retraction of the guidewire 34 into the open region 40, which would require re-setting the guidewire 34 into the needle assembly 12. In an embodiment as seen in FIG. 3B, the guidewire 34 has been fully retracted and a tip 150 of the guidewire is partially within the handle connector 24.

With the guidewire tip 150 in this retracted or storage position, the lumen formed in the needle 14 and the needle connector 16 is empty, i.e., not occupied by the guidewire 34. Additionally, this space is vented by way of vents 52 formed at the periphery of the opening 254 of the handle connector 24 where it surrounds the guidewire 34.

In an embodiment, at least one of the needle connector 16 and the handle connector 24 has a vented chamber formed therein to maintain ambient pressure in the vented chamber. In one embodiment, the insertion device 10 comprises a transparent region or a translucent region 55, 56 to provide a visual indication of blood entering such region. In an embodiment, the vented chamber 54 comprises the transparent region or a translucent region 55. In an embodiment, the insertion device 10 is configured to provide a visible indicator of a placement of the needle tip 18. In an embodiment, the visible indicator is provided without the use of a pressure reducing component such as a syringe or vacuum generating device.

The vented chamber 54 may be the space between the male luer connector for the needle hub 16 and the advancer handle 22. This chamber 54 is considered vented because there is an open gap between the guidewire 34 and the hole 254 through the advancer handle 22, meaning it is not sealed. The vented chamber 54 is not directly connected to the dispensing tube or the plug.

In one embodiment, the venting of air or gas in front of the blood occurs in part because the passageway between the advancer and the guidewire is not intentionally sealed. A small gap exists between the internal diameter of the needle and the guidewire, when the guidewire is in the needle passageway, and a larger space is present in the needle if the guidewire is retracted into the male luer locking connector (i.e., bonded to the advancer). Blood “flashback” into the semi-clear visualization area of the connector occurs when the user advances the needle into a blood vessel of the the patient with the guidewire fully retracted. Leak-back of blood through the needle is a normal and desirable feature of vascular access for many techniques. It (1) confirms the needle is actually in a blood vessel, and (2) is an indicator of correct blood vessel location as arterial blood flows out much faster and typically redder than venous blood, which is at lower pressure.

In an embodiment, the needle 14 has an inner diameter in the range of 0.8 mm and 1.25 mm. In an embodiment, the guidewire 34 has an outer diameter in the range of 0.7 mm and 1.1 mm. In an embodiment, the gap between the inner diameter of the needle 14 and the outer diameter of the guidewire is in the range between 0.10 mm and 0.16 mm. By way of example, an 18 ga. needle has an inner diameter of about 0.04 inches (1.02 mm) and a typical guidewire 34 may have an outer diameter of about 0.035 inches (0.89 mm). This leaves a gap of about 0.005 inches (0.13 mm). The inventors have found that leaving this lumen open by retracting the guidewire 34 at least partially past the distal end of the handle connector 24 allows for backflow of fluid from a target body region, even where the target body region is a low pressure region such as a vein.

As a result, there is no need for a syringe or other vacuum source to encourage backflow from the target body region by drawing negative pressure. Instead, even low pressure fluid can flow into the chamber 54 of the handle connector 24. As noted above, this handle connector 24 may include transparent or translucent region 55 (e.g., of a transparent or translucent plastic, elastomeric, or glass material) to allow visualization of the presence of fluid therein. In one embodiment, the transparent or translucent region 55 of the handle connector 24 may comprise a plastic material. This flow can be considered passive flow, and ambient pressure of the chamber 54 along with the venting through vent 52 allows this backflow, whereas a syringe would tend to create an unvented region that would tend to prevent or reduce backflow without actuating the syringe to create a vacuum or reduced pressure region. That is, the device 10 may be operated without the use of a pressure reducing component such as a syringe or vacuum generating device.

In general, parts described herein as separate may likewise be made unitary. For example, while the handle portion 22 has been described in two parts as including a separate advancer frame 26 and the guidewire tube 28, these two parts may in principle be made as a single component, or may be glued together to make them act as a unitary component.

In an embodiment, the insertion device 10 comprises a locking collar that is configured to releasably connect the needle connector 16 and the frame connector portion 24. The locking collar is configured to constrain relative rotation between the frame connector portion 24 and the needle connector 16 so as to maintain the needle assembly 12 in a predetermined rotational orientation relative to the frame 22 during use. In an embodiment, the locking collar includes interlocking members that are configured to engage with portions on surfaces of the needle connector 16. The locking collar may have a configuration which is the same as or similar to Merit Medical Part Number 101047001 (e.g., see, https://meritoem. com/wp-content/uploads/2015/11/19-101047001MALE-FEMALE-LUER-LOCK-CONNECTOR.pdf), or a Luer-Lock connection as described in Anesthesia Patient Safety Foundation Newsletter, Volume 36, No. 3, October 2021, for example (e.g., see, https://www.apsf.org/article/managing-luer-connections), which are incorporated by reference in their entirety herein.

In an embodiment, the needle tip 18 includes the pointed end 62 and the beveled sides 64 adjacent the pointed end 62. In the predetermined rotational orientation during use, the beveled sides 64 of the needle tip 18 are configured to point away from the needle assembly 12.

In an embodiment, the predetermined rotational orientation during use in which the beveled sides 64 of the needle tip 18 are configured to point away from the needle assembly 12 may be referred to as a bevel-up configuration.

In one embodiment, when advancing the needle assembly 12, especially through more dense or in-elastic tissues such as those in Chronic Kidney Disease (CKD) patients, the vein or vascular structure may undergo increased compression, resulting in ovalizing or flattening of the vein or vascular structure. The resulting flattening increases the likelihood of over penetrating the vessel and puncturing the opposite (posterior) wall, which may then result in undesired bleeding. In one embodiment, the asymmetrical echogenic structure 20 helps prevent this by producing a slight increase in penetration resistance/force upon contacting the anterior (first) vessel wall.

In one embodiment, the design of the asymmetric tapered bulb 20 optimizes performance by reducing the force necessary for initial needle advancement through tissue, while maintaining sufficient transition taper to produce a detectable increase in vessel wall penetration force upon tip entry. The steeper trailing profile of the bulb further resists unintended backward movement, helping to stabilize needle position during guidewire advancement.

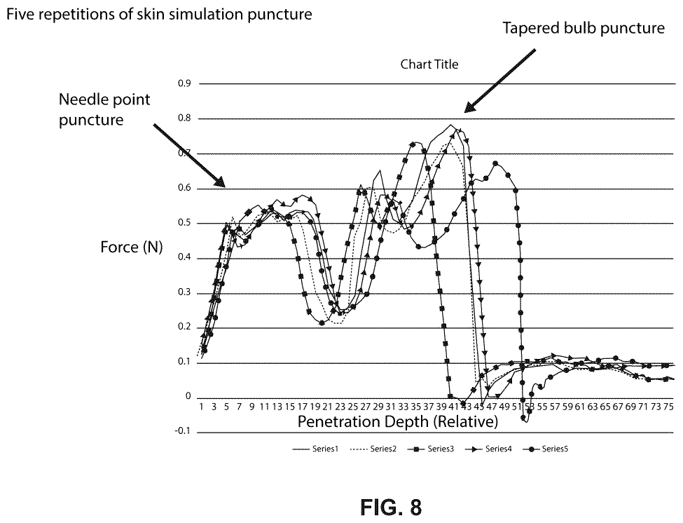

FIG. 8 shows a graphical representation of how the needle assembly 12 with the asymmetrical echogenic structure 20 penetrates a silicone sheet (skin simulation). FIG. 8 shows changes in force that occur as the needle tip 18 and the asymmetrical echogenic structure 20 pass through the surface (e.g., vessel (artery or vein) wall). The graph shows a relationship between the penetration depth in millimeters (shown along the X-axis) and the force in Newtons (shown along the Y-axis) for five puncture trials using the asymmetrical echogenic structure 20 disposed on the needle assembly 12 and the silicone sheet (e.g., for skin simulation).

For each puncture trial, the curve initially rises to a peak that represents the force required for the needle tip 18 to puncture the surface. There is a sharp (force) drop after this initial penetration. As the penetration of the needle assembly 12 continues, there is a second force increase as the asymmetrical echogenic structure 20 contacts the skin or vessel wall. This second force increase results in a deformation of the vessel wall visible on the ultrasound, as well as keeping the needle assembly 12 from accidentally over-penetration the vessel and penetrating the opposite wall, which can cause undesirable bleeding (blood extravasation).

In accordance with various embodiments, the echogenic E bulb may be applied to many different medical needles used for percutaneous (through-the-skin) procedures or other tissue-penetrating procedures performed under ultrasound or fluoroscopic guidance. The echogenic bulb enhances the ultrasound signal, making it more visible under ultrasound than the needle on which it is placed. The echogenic bulb also provides a second force peak when penetrating a vessel wall, organ wall (such as the pericardium, trachea, embryonic sac, etc.), membrane wall, or other tissue that a needle may penetrate.

The echogenic bulb may be attached to needles across the full range of medical needle sizes, including sizes from 7 gauge (Ga) to 34 gauge (Ga) on the Birmingham Gauge Scale. The most common needle sizes for use with the echogenic bulb are typically in the range of 12 Ga to 25 Ga.

In one embodiment, the bulb diameter is shown as BD in FIG. 7 and the needle size (i.e., needle cannula outer diameter) is shown as NOD in FIG. 7. In this particular embodiment, the needle size (i.e., needle cannula outer diameter) shown as NOD in FIG. 7 is same as the widest diameter portion WDP of the bulb 20.

In various embodiments, a needle may optionally have more than one bulb. In one embodiment, the needle may have thereon two or more bulbs of different configurations. These bulbs could be separated by various distances. In some embodiments, those distances may be proportional to the bulb length. For example, in some embodiments, the distances are in the range of 0.5 to 3 times the bulb length. This separation can help indicate penetration of a wall, lining or tissue boundary.

In one embodiment, the multiple bulbs may have different diameters. In one embodiment, the bulbs may have the same diameter (i.e., symmetric or asymmetric). In one embodiment, the bulbs may have increasing diameter going away from the needle tip (e.g., bulb-2 is larger diameter than bulb-1).

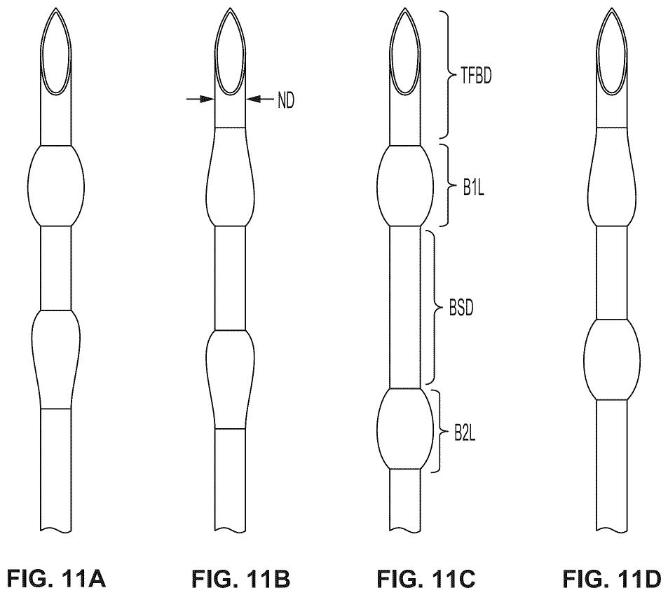

Examples of variations (shown in FIGS. 11A-11D) include configurations with two or more bulbs having symmetric and/or asymmetric shapes. The distance from the needle tip to the first bulb TFBD may vary, as may the separation between the first and second bulbs BSD, as well as any additional bulbs. Bulb lengths may also vary, with bulb-1 (B1L), bulb-2 (B2L), and subsequent bulbs each having different lengths. In FIGS. 11A and 11C, the forwardmost bulb is symmetric, and in 11C, both the forward and rearward bulbs are symmetric. In FIGS. 11D and 11C, the rearward bulb is symmetric. In FIGS. 11D and 11B, the forward bulb is asymmetric, with the largest diameter portion of the bulb being closer to the rearward end of the needle than the needle tip. In FIGS. A And 11B, the rearward bulbs have the largest diameter positioned closer to the needle tip than the rearward end of the needle.

Several dimensional variables of the echogenic bulb may be adjusted in accordance with various embodiments. For example, in one embodiment, the diameter of the echogenic bulb may be between 1.2 and 2.5 times the outer diameter of the needle cannula. For example, for an 18 Ga needle having an outer diameter of approximately 1.27 mm, the Echogenic bulb diameter may range from 1.52 mm to 3.18 mm.

In other embodiments, the length of the echogenic bulb may be adjusted. For example, in various embodiments, the length may be between 1.0 and 3.0 times the echogenic bulb diameter. Correspondingly, in some embodiments, the echogenic bulb length may be between 2.0 and 4.0 times the outer diameter of the needle cannula.

In other embodiments, the relationship between the location of the maximum diameter of the echogenic bulb and the front edge of the echogenic bulb nearest the needle tip may be adjusted. For example, in one embodiment, such as that in FIGS. 11B and 11B, the maximum diameter for the forward most bulb closest to the needle tip may be positioned such that between 65% to 85% of the bulbs length is positioned between the maximum diameter position and the end of the bulb closest to the rearward end of the needle and between 35% to 15% of the bulbs length is positioned between the maximum diameter position and the end of the bulb closest to the needle tip. In another embodiment, the maximum diameter for the forwardmost bulb is positioned such that between 55% to 80% of the bulbs length is positioned between the maximum diameter position and the end of the bulb closest to the rearward of the needle and between 45% to 20% of the bulbs length is positioned between the maximum diameter position and the end of the bulb closest to the needle tip. It should be appreciated that in FIGS. 11A and 11B, the rearward most bulb may have these rations inverted (with the maximum diameter being closer to the tip than the rearward end of the needle.

As used herein, enhanced echogenicity refers to one or both of the following effects:
the location of the bulb being easier to visualize than the needle cannula alone, and the effect of the bulb on surrounding tissue being more readily visible on ultrasound than the needle cannula alone. Enhanced haptics refers to increased tactile feedback, such that the needle provides greater touch sensitivity or tactile sensation as the bulb passes through tissue, a vessel wall, or other body tissue.

In one embodiment, the needle includes at least two bulbs, with bulb-1 having an asymmetric shape and bulb-2 having a symmetric shape. In one embodiment, the distance from the needle tip to bulb-1 (TFBD) may be between 0.5 and 2.0 times the length of bulb-1. In one embodiment, the separation between bulb-1 and bulb-2 (BSD) may be between 0.5 and 3 times the length of bulb-1. In one embodiment, the length of bulb-1 (B1L) may be between 2 and 4 times the needle diameter (ND), and the length of bulb-2 (B2L) may likewise be between 2 and 4 times the needle diameter (ND).

In one embodiment, as shown in FIG. 11(D), bulb-1 is asymmetric and includes a tapered leading edge configured to pass more easily through tissue to a blood vessel or other organ wall, such as the pericardium. Bulb-1 may be visualized on ultrasound compressing the vessel wall and then felt to “pop” through the vessel wall, providing enhanced haptic feedback. Bulb-2 then limits over-penetration through the vessel wall and remains in contact with the vessel wall, where it is visible due to enhanced echogenicity and felt due to increased resistance to further advancement. In this configuration, the vessel wall is retained between bulb-1 and bulb-2, such that retracting or advancing the needle results in corresponding movement of the vessel wall that is more easily seen on ultrasound and felt as resistance. Asymmetric bulbs may be oriented with the taper facing the direction of desired lower-force advancement or retraction.

In one embodiment, a method of inserting a guidewire is provided. The method comprises providing a needle assembly having a distal end with a needle tip and a needle connector; releasably connecting a handle portion to the needle assembly by mating/connecting a handle connector portion of the handle portion with the needle connector of the needle assembly; providing a guidewire tube connected to the handle portion, the guidewire tube being coiled to store a guidewire and having an uncoiled length that is longer than a coiled circumference; and positioning the handle portion and the coil of the guidewire tube such that the handle portion and the coil of the guidewire tube generally extend along a common plane and an axis through a center of the coil of the guidewire tube is substantially perpendicular to the common plane.

In one embodiment, a method of inserting a guidewire is provided. The method comprises providing a needle assembly having a distal end with a needle tip and a needle connector; releasably connecting a handle portion to the needle assembly by mating/connecting a handle connector portion of the handle portion with the needle connector of the needle assembly, wherein at least one of the needle connector and the handle connector portion is sufficiently transparent such that a fluid present therein is visible; observing fluid within the at least one transparent connector; and providing a guidewire tube connected to the handle portion, the guidewire tube being coiled to store a guidewire.

In one embodiment, a method of inserting a guidewire is provided. The method comprises providing a needle assembly having a distal end with a needle tip and a needle connector; releasably connecting a handle portion to the needle assembly by mating/connecting a handle connector portion of the handle portion with the needle connector of the needle assembly; and providing a guidewire tube connected to the handle portion, the guidewire tube being coiled to store a guidewire, wherein a diameter of coils of the coiled guidewire tube is between about 2 inches and about 4 inches.

In one embodiment, a method of inserting a guidewire is provided. The method comprises providing a needle assembly having a distal end with a needle tip and a needle connector; releasably connecting a handle portion to the needle assembly by mating/connecting a handle connector portion of the handle portion with the needle connector of the needle assembly, wherein at least one of the needle connector and the handle connector portion includes a vented chamber; maintaining ambient pressure in the vented chamber; providing a guidewire tube connected to the handle portion; and providing a visual indication of blood entering a transparent or translucent region of the insertion device.

In one embodiment, a method of inserting a guidewire is provided. The method comprises providing a needle assembly having a distal end with a needle tip and a needle connector; providing an echogenic structure disposed on the needle assembly at a predetermined distance from the needle tip, the echogenic structure comprising a bulb having an asymmetric shape along a longitudinal axis of the needle assembly, the bulb having a proximal end and a distal end, the distal end being closer to the needle tip than the proximal end, and a widest diameter portion of the bulb is closer to the proximal end than the distal end; releasably connecting a frame portion, which is configured to be manually engaged by a user, to the needle assembly by mating/connecting a frame connector portion of the frame portion with the needle connector of the needle assembly; and providing a guidewire tube connected to the frame portion, the guidewire tube coiled to store a guidewire.

In one embodiment, a method of inserting a guidewire is provided. The method comprises providing a needle assembly having a distal end with a needle tip and a needle connector; releasably connecting a frame portion, which is configured to be manually engaged by a user, to the needle assembly using a locking collar that releasably connects a frame connector portion of the frame portion and the needle connector of the needle assembly; constraining relative rotation between the frame connector portion of the frame portion and the needle connector of the needle assembly using the locking collar so as to maintain the needle assembly in a predetermined rotational orientation relative to the frame during use; and providing a guidewire tube connected to the frame portion, the guidewire coiled to store a guidewire.

In one embodiment, a method of inserting a guidewire is provided. The method comprises providing a needle assembly having a distal end with a needle tip and a needle connector; releasably connecting a frame portion, which is configured to be manually engaged by a user, to the needle assembly by mating/connecting a frame connector portion of the frame portion with the needle connector of the needle assembly, and providing a guidewire tube connected to the frame portion, the guidewire tube coiled to store a guidewire, wherein the frame has a maximum thickness portion along a plane perpendicular to the guidewire, wherein a diameter of coils of the guidewire tube is between about 2 inches and about 4 inches, and wherein the guidewire tube lies within the imaginary thick plane defined by the maximum thickness portion.

In one embodiment, a method of inserting a guidewire is provided. The method comprises providing a needle assembly having a distal end with a needle tip and a needle connector; releasably connecting a frame portion, which is configured to be manually engaged by a user, to the needle assembly by mating/connecting a frame connector portion of the frame portion with the needle connector of the needle assembly, the frame portion including an open portion between the frame connector portion of the frame portion and a proximal end of the frame portion; providing a guidewire tube connected to the frame portion, the guidewire tube coiled to store a guidewire; and pinching the guidewire through the open portion of the frame portion between a user's thumb and finger to control advancement or retraction of the guidewire. In one embodiment, a method of inserting a guidewire is provided. The method comprises providing a needle having a distal end with a needle tip and a proximal end; providing a first echogenic structure disposed on the needle, spaced at a first distance from the needle tip; and providing a second echogenic structure disposed on the needle, the second echogenic structure spaced a second distance from the needle tip, wherein the second distance is greater than the first distance.

The descriptions above are intended to be illustrative, not limiting. Thus, it will be apparent to one skilled in the art that modifications may be made as described without departing from the scope of the claims set out below. Where ranges are disclosed, the range may be inclusive or exclusive of its endpoints. That is, a range of “from 1-10” may be interpreted as “greater than 1 to less than 10” or “from 1 to 10 inclusive.”

What is claimed is:

1. An insertion device comprising:
a needle assembly having a distal end with a needle tip and needle connector;
a handle portion releasably connectable to the needle assembly, the handle portion comprising a handle connector portion that is configured to mate with the needle connector; and
a guidewire tube connected to the handle portion, the guidewire tube coiled to store a guidewire, the guidewire tube having an uncoiled length that is longer than its coiled circumference, wherein the coil of the guidewire tube and the handle portion generally extend along a common plane,
wherein an axis through a center of the coil of the guidewire tube is substantially perpendicular to the common plane.

2. The insertion device of claim 1, further comprising an asymmetric echogenic structure disposed at a predetermined distance from the needle tip.

3. The insertion device of claim 2, wherein the echogenic structure is a bulb of material having an echogenicity different from an echogenicity of a material of the needle tip.

4. The insertion device of claim 2, wherein the echogenic structure is a bulb of material having an echogenicity same as an echogenicity of a material of the needle tip.

5. The insertion device of claim 1, further comprising a plurality of clips disposed about the circumference of the guidewire tube and configured and arranged to secure adjacent coils to each other.

6. The insertion device of claim 1, wherein the handle portion further comprises an open portion between the handle connector portion and a proximal end of the handle portion, and
wherein the guidewire extends across the open portion from the proximal end of the handle portion to the handle connector portion and at least partly into the needle assembly.

7. The insertion device of claim 5, wherein the open portion includes a platform portion against which a user can push the guidewire to advance or retract it.

8. The insertion device of claim 1, wherein the guidewire tube includes an end having a stop configured to prevent retraction of the guidewire past the handle connection portion of the handle portion.

9. The insertion device of claim 8, wherein a total length of the guidewire is selected to be greater than a total length of the handle portion measured between the connector portion and a proximal end of the handle portion plus a total length of the guidewire tube.

10. The insertion device of claim 9, wherein the total length of the guidewire is further selected to be less than a total length of the handle portion measured between the connector portion and the proximal end of the handle portion plus a total length of the guidewire tube plus a total length of the needle assembly.

11. The insertion device of claim 1, wherein a diameter of the coiled guidewire tube is in the range between 2 inches and 4 inches.

12. An insertion device comprising:
a needle assembly having a distal end with a needle tip and a needle connector;
a handle portion releasably connectable to the needle assembly, the handle portion comprising a handle connector portion that is configured to mate with the needle connector,
wherein at least one of the needle connector and the handle connector is sufficiently transparent such that a fluid present therein is visible; and
a guidewire tube connected to the handle portion, the guidewire tube coiled to store a guidewire.

13. The insertion device of claim 12, wherein the needle connector further comprises a vent such that pressure in a chamber formed therein is at ambient pressure such that a low pressure fluid present at the needle tip is able to backflow into the chamber.

14. An insertion device comprising:
a needle assembly having a distal end with a needle tip and a needle connector;
a handle portion releasably connectable to the needle assembly, the handle portion comprising a handle connector portion that is configured to mate with the needle connector; and
a guidewire tube connected to the handle portion, the guidewire tube coiled to store a guidewire, a diameter of coils of the coiled guidewire tube is in the range between 2 inches and 4 inches.

15. The insertion device of claim 14, wherein the handle portion includes an opening into which a finger of a user can be inserted.

16. An insertion device comprising:
a needle assembly having a distal end with a needle tip and a needle connector;
a handle portion releasably connectable to the needle assembly, the handle portion comprising a handle connector portion that is configured to mate with the needle connector;
at least one of the needle connector and the handle connector having a vented chamber formed therein to maintain ambient pressure in the vented chamber; and
a guidewire tube connected to the handle portion;
wherein the insertion device comprises a transparent or translucent region to provide a visual indication of blood entering such region.

17. The insertion device of claim 16, wherein the vented chamber comprises the transparent or translucent region.

18. An insertion device comprising:
a needle assembly having a distal end with a needle tip and a needle connector;
a frame portion configured to be manually engaged by a user and releasably connectable to the needle assembly, the frame portion comprising a frame connector portion that is configured to mate with the needle connector;
a guidewire tube connected to the frame portion, the guidewire tube coiled to store a guidewire; and
an echogenic structure disposed on the needle assembly at a predetermined distance from the needle tip,
wherein the echogenic structure comprises a bulb having an asymmetric shape along a longitudinal axis of the needle assembly,
wherein the bulb having a proximal end and a distal end, the distal end being closer to the needle tip than the proximal end, and
wherein a widest diameter portion of the bulb is closer to the proximal end than the distal end.

19. The insertion device of claim 18, wherein the needle assembly includes a needle shaft extending between the needle tip and the needle connector,
wherein the needle shaft is configured to receive the guidewire therethrough, and
wherein the bulb is disposed on the needle shaft between the needle tip and the needle connector.

20. The insertion device of claim 19, wherein the bulb has a maximum diameter between 1.7 and 2.1 mm at the widest diameter portion perpendicular to the longitudinal axis of the needle assembly.

21. The insertion device of claim 20, wherein the maximum diameter of the bulb is disposed at a distance in the range between 0.8 mm and 1.4 mm from the proximal end of the bulb, and
wherein the widest diameter portion of the bulb is shaped and dimensioned to engage with tissue portions around a target body region to resist retraction of the needle shaft from the target body region.

22. The insertion device of claim 21, wherein at least a portion of the bulb is positioned at a distance between 1 mm and 4 mm from the needle tip.

23. The insertion device of claim 22, wherein the bulb has an overall length between 3.5 mm and 4.5 mm measured along the longitudinal axis of the needle assembly.

24. The insertion device of claim 23, wherein a ratio of the overall length of the bulb to the maximum diameter of the bulb is in the range between 2.06 mm and 2.14 mm.

25. The insertion device of claim 19, wherein the echogenic structure is made of a material having an echogenicity different from an echogenicity of a material of the needle shaft.

26. The insertion device of claim 18, wherein the bulb is formed from a UV-cured polymeric material containing echogenic particles selected from the group consisting of tungsten, stainless steel, or titanium dioxide.

27. The insertion device of claim 18, wherein the guidewire tube has an uncoiled length that is longer than its coiled circumference, a diameter of the coiled guidewire tube is in the range between 2 inches and 4 inches.

28. An insertion device comprising:
a needle assembly having a distal end with a needle tip and a needle connector;
a frame portion configured to be manually engaged by a user and releasably connectable to the needle assembly, the frame portion comprising a frame connector portion;
a guidewire tube connected to the frame portion, the guidewire tube coiled to store a guidewire; and
a locking collar that is configured to releasably connect the needle connector and the frame connector portion,
wherein the locking collar is configured to constrain relative rotation between the frame connector portion and the needle connector so as to maintain the needle assembly in a predetermined rotational orientation relative to the frame during use.

29. The insertion device of claim 28, wherein the locking collar includes interlocking members that are configured to engage with portions on surfaces of the needle connector.

30. The insertion device of claim 28, wherein the needle tip includes a pointed end and beveled sides adjacent the pointed end, and
wherein, in the predetermined rotational orientation during use, the beveled sides of the needle tip are configured to point away from the needle assembly.

31. The insertion device of claim 28, wherein the guidewire tube has an uncoiled length that is longer than its coiled circumference, a diameter of the coiled guidewire tube is in the range between 2 inches and 4 inches.

32. An insertion device comprising:
a needle assembly having a distal end with a needle tip and a needle connector;
a frame portion configured to be manually engaged by a user and releasably connectable to the needle assembly, the frame portion comprising a frame connector portion configured to mate with the needle connector; and
a guidewire tube connected to the frame portion, the guidewire tube coiled to store a guidewire, a diameter of coils of the coiled guidewire tube is between 2 and 4 inches, the frame having a maximum thickness portion along a plane perpendicular to the guidewire, wherein the guidewire tube lies within an imaginary thick plane defined by the maximum thickness portion.

33. The insertion device of claim 32, wherein the diameter of the coiled guidewire tube is between 2 and 3 inches.

34. The insertion device of claim 32, wherein the diameter of the coiled guidewire tube is between 2.5 and 3 inches.

35. The insertion device of claim 32, wherein the diameter of the coiled guidewire tube is 2.5 inches.

36. The insertion device of claim 32, wherein the diameter of the coiled guidewire tube is 3 inches.

37. An insertion device comprising:
a needle assembly having a distal end with a needle tip and a needle connector;
a frame portion configured to be manually engaged by a user and releasably connectable to the needle assembly, the frame portion comprising a frame connector portion configured to mate with the needle connector; and
a guidewire tube connected to the frame portion, the guidewire tube coiled to store a guidewire,
wherein the frame portion includes an open portion between the frame connector portion and a proximal end of the frame portion, and
wherein the open portion is configured to allow for a user to pinch the guidewire between a user's thumb and a user's finger for controlled advancement or retraction of the guidewire.

38. The insertion device of claim 37, wherein the length of the open portion is between 10 mm and 50 mm.

39. The insertion device of claim 37, wherein the length of the open portion is between 20 mm and 40 mm.

40. The insertion device of claim 37, wherein the frame portion includes a mating portion.

41. The insertion device of claim 40, wherein the distance between the mating portion and the frame connector portion is in the range between 56 mm and 84 mm.

42. The insertion device of claim 37, wherein the open portion includes a platform portion against which a user can push the guidewire to advance or retract guidewire.

43. The insertion device of claim 42, wherein the platform portion includes one or more protrusions extending in a direction perpendicular to a surface of the platform portion and perpendicular to an axis of the guidewire.

44. The insertion device of claim 37, wherein the open portion includes a platform portion having one or more protrusions configured to facilitate advancement and retraction of the guidewire,
wherein the platform portion is positioned such that the user's thumb is able to engage the guidewire from above and apply pressure against the platform portion to advance or retract the guidewire, and
wherein the one or more protrusions are configured to frictional contact between the guidewire and the platform portion by limiting an area of surface contact therebetween.

45. The insertion device of claim 44, wherein the open portion is configured to permit both thumb-slide actuation and pinch actuation of the guidewire.

46. The insertion device of claim 37, wherein the user's finger is a middle finger.

47. The insertion device of claim 37, wherein the user's finger is an index finger.

48. The insertion device of claim 37, wherein the guidewire extends across the open portion from a proximal end of the frame portion to the frame connector portion and at least partly into the needle assembly.

49. An needle device, comprising:
a needle having a distal end with a needle tip and a proximal end;
a first echogenic structure disposed on the needle, spaced at a first distance from the needle tip,
a second echogenic structure disposed on the needle, the second echogenic structure spaced a second distance from the needle tip, wherein the second distance is greater than the first distance.

50. A needle device accordance to claim 49, wherein the first echogenic structure and/or the second echogenic structure comprises an asymmetric bulb shape.

51. A needle device according to claim 50, wherein a first echogenic structure is asymmetric and has its widest diameter positioned closer to the needle tip than the rear end of the need.

52. A needle device according to claim 50, wherein a first echogenic structure is asymmetric and has its widest diameter positioned closer to the rearward end of the needle than the needle tip.

53. A needle device according to claim 51, wherein the first echogenic structure is disposed closer to the rear end of the needle than the second echogenic structure.

54. A needle device according to claim 52, wherein the first echogenic structure is disposed closer to the needle tip than the second echogenic structure.

55. A needle device according to claim 49, further comprising a needle connector at the proximal end of the needle configured to connect the needle device to a frame.
